(19) United States
(12) Patent Application Publication
(10) Pub. No.: US 2025/0231143 A1
(43) Pub. Date: Jul. 17, 2025

(54) IN-SITU ANALYSIS OF METAL SPECIES FOR PROCESSING CHAMBERS

(71) Applicant: Applied Materials, Inc., Santa Clara, CA (US)

(72) Inventor: Jiansheng Wang, Union City, CA (US)

(21) Appl. No.: 19/015,451

(22) Filed: Jan. 9, 2025

Related U.S. Application Data
(60) Provisional application No. 63/620,494, filed on Jan. 12, 2024.

(51) Int. Cl. G01N 27/68; G01N 33/00; G01N 33/2028

(52) U.S. Cl. CPC G01N 27/68; G01N 33/0036; G01N 33/2028

(57) ABSTRACT

Systems and methods are provided for analysis of metal contaminants within a processing chamber. Such systems and methods can include a vacuum system and valve attached to a processing chamber. The vacuum system and valve can effect a reduction in a pressure within a sampling chamber, an opening, while a process is in effect within a processing chamber connected to the sampling chamber, of a first fluid connection from the sampling chamber to the processing chamber such that a fluid ingresses from the processing chamber into the sampling chamber, a closing of the first fluid connection, an opening a second fluid connection from the sampling chamber to an inductively coupled plasma mass spectrometer (ICP-MS) such that the fluid egresses from the sampling chamber into the ICP-MS, an identification, via the ICP-MS, of a presence of trace elements within the fluid, and responsive to the identified presence of trace elements, performance of a corrective action associated with the processing chamber.

### RELATED APPLICATIONS

This patent application claims the benefit under 35 U.S.C. § 119(e) of U.S. Provisional Application No. 63/620,494, filed Jan. 12, 2024, which is incorporated by reference herein.

### TECHNICAL FIELD

Embodiments of the present disclosure relate generally to analysis of contaminants within a processing chamber, and in particular to in-situ analysis of metal species contaminants within a processing chamber.

### BACKGROUND

Metal contamination on semiconductor wafers is a long-standing challenge within the semiconductor manufacturing industry. This type of contamination often includes metal particles (or metal containing species in gas form, such as sublimation under vacuum and/or under high temperature). In general, particles have been widely studied and understood that these particles can originate from various sources and confound quality and reliability of manufactured semiconductor devices. However, only limited understanding on the metal containing species in gas form(s).

Such metal particles, or nanoparticles, can originate from various sources throughout the manufacturing process. These varied sources, may make contamination difficult to trace, control, and eliminate. For example, such particles may originate from any of the equipment, materials, chemicals, and/or gases used within a processing chamber of a manufacturing process. In some cases, corrosion under the harsh conditions inside a chamber can lead to the release of metal particles. In some cases, coatings used within a chamber can deteriorate and peel off, forming additional particulate matter.

Notably, these metal particles can sometimes become present in gaseous forms during the production stages. Thus, contaminant particles such as stainless steel, ceramic aluminum, or other susceptible materials, can be present and impact the working gas within a processing chamber. Understanding and controlling this type of metal particle contamination is critical in maintaining the high standards in semiconductor manufacturing.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

In some aspects, a method is provided. In some aspects, the method includes reducing a pressure within a sampling chamber, opening, while a process is in effect within a processing chamber connected to the sampling chamber, a first fluid connection from the sampling chamber to the processing chamber such that a fluid ingresses from the processing chamber into the sampling chamber, closing the first fluid connection, opening a second fluid connection from the sampling chamber to a spectrometric device such as an inductively coupled plasma mass spectrometer (ICP-MS) such that the fluid egresses from the sampling chamber into the spectrometric device, identifying, via the spectrometric device, a presence of trace elements within the fluid, and responsive to the identified presence of trace elements, performing a corrective action associated with the processing chamber.

In some aspects, a system is provided. In some aspects, the system includes a processing chamber, a spectrometric device to identify a presence of trace elements within a fluid from the processing chamber, and a valve configured to connect the processing chamber to a sampling chamber in a first valve setting to cause the fluid to ingress from the processing chamber into the sampling chamber and to connect the sampling chamber to the spectrometric device in a second valve setting to cause the fluid to egress from the sampling chamber into the spectrometric device.

In some aspects, a non-transitory computer readable storage medium is provided. In some aspects, the non-transitory computer readable storage medium includes instructions that, when executed by a processing device, causes the processing device to perform operations including reducing a pressure within a sampling chamber, opening, while a process is in effect within a processing chamber connected to the sampling chamber, a first fluid connection from the sampling chamber to the processing chamber such that a fluid ingresses from the processing chamber into the sampling chamber, closing the first fluid connection, opening a second fluid connection from the sampling chamber to an inductively coupled plasma mass spectrometer (ICP-MS) such that the fluid egresses from the sampling chamber into the ICP-MS, identifying, via the ICP-MS, a presence of trace elements within the fluid, and responsive to the identified presence of trace elements, performing a corrective action associated with the processing chamber.

### BRIEF DESCRIPTION OF THE DRAWINGS

Aspects and implementations of the present disclosure will be understood more fully from the detailed description given below and from the accompanying drawings, which are intended to illustrate aspects and implementations by way of example and not limitation.

FIG. 1 illustrates a top-down view of an exemplary embodiment of a substrate manufacturing system, according to some embodiments of the present disclosure.

FIG. 2 illustrates a schematic view of an exemplary contaminant analysis system and exemplary substrate processing chamber of FIG. 1, according to some embodiments of the present disclosure.

FIG. 3A illustrates a first exemplary configuration of the contaminant analysis system of FIG. 2, according to some embodiments of the present disclosure.

FIG. 3B illustrates a second exemplary configuration of the contaminant analysis system of FIG. 2, according to some embodiments of the present disclosure.

FIG. 4 is a flow diagram of an example method of performing in-situ analysis of metal species within a semiconductor processing chamber, in accordance with one embodiment of the present disclosure.

FIG. 5 illustrates an embodiment of a diagrammatic representation of a computing device associated with a substrate manufacturing system, in according to some embodiments of the present disclosure.

### DETAILED DESCRIPTION

Analysis of metallic contaminants in semiconductor manufacturing is predominantly conducted offline. Typically, analysis is centered around the examination of a process chamber and/or various components within the process chamber while the process chamber is out of service. Such components that may be tested include the shower head, ESC, liners, the evacuation system, or gas lines, etc. For example, a test recipe may be performed to cause metal particles to settle on a test wafer, and the test wafer may then be analyzed for metal contaminants. Such testing may be performed either before or after chamber processes to detect any anomalies.

If and when such analysis detects metal contamination on a wafer, the remediation process often involves waiting for a scheduled maintenance period. During this downtime, the chamber is often opened, and the suspected part(s) carefully removed for further examination.

Analysis of the extracted part often involves dissolving any metallic contaminants into a solution and processing the solution through an inductively coupled plasma mass spectrometry (ICP-MS) system. An ICP-MS system can be used to detect and quantify trace elements, such as metal particles, in gas samples. The process involves nebulizing the gas or liquid sample (e.g., into an aerosol), which is then introduced into a plasma torch to atomize and ionize the sample. Ions are filtered through quadrupoles in the mass spectrometer and thus different elements or isotopes can be detected and measured. Additionally, a single particle identification component, i.e., a single particle ICP-MS (sp-ICP-MS), can be included to offer further detection of nanoparticles in addition to trace metals. This method offers high sensitivity and accuracy, even in parts per trillion, ensuring reliable quantification of trace elements within a gas samples.

One significant issue with such analysis is the time-consuming nature of this “off-line” process. The wait for a maintenance window to access and analyze the chamber parts adds considerable delay to the identification and resolution of contamination issues.

Furthermore, since the analysis is done after the fact, there is a lag between the instance of contamination and its detection. This delay complicates the process of tracing contaminations back to root cause(s). E.g., as production continues, more variables and changes occur in the manufacturing environment, making it increasingly difficult to pinpoint the exact source and time of contamination.

In such a way, delays from such methods of contamination analysis methods can lead to extended periods of manufacturing with compromised quality, potentially affecting a large volume of semiconductor products. Accordingly, such an offline analysis method's slow paced and retrospective nature present considerable limitations in a fast-paced manufacturing environment.

Thus, embodiments described herein address the above, and other challenges, by introducing systems and methods for analysis of metallic contamination in real-time, or in-situ, during use of a processing chamber on production substrates. In embodiments, the proposed systems and methods seek to enhance precision and response time when tracing processing chamber contaminations.

In embodiments, a proposed system or contamination analysis can include a sampling chamber and a spectrometric device such as an ICP-MS system (including an sp-ICP-MS) for identifying and measuring contamination within a processing chamber, in-situ. Other types of spectrometric devices may also be used, such as a time-of-flight mass spectrometry (TOF-MS) device or a gas chromatography-mass spectrometry (GC-MS) device. In embodiments, the proposed analysis system may include a valve for operatively connecting and isolating the sampling chamber from both the main processing chamber, and the spectrometric device. To begin analysis, the sampling chamber may be isolated from the processing chamber, and the sampling chamber pressure may be reduced, or lowered, to a pre-determined level. A fluid connection between the sampling chamber and processing chamber may be opened such that working fluid (e.g., gas which may contain contaminant metal particles) may ingress into the sampling chamber. Afterwards, the fluid connection between the sampling chamber and processing chamber (and between the sampling chamber and a vacuum pump) may be closed, and a separate fluid connection between the sampling chamber and an ICP-MS system (and between the sampling chamber and an inert gas source) may be opened. The working fluid may be flowed into the spectrometric device by an inert gas from the inert gas source. The spectrometric device system may then identify and quantify any contaminants within the sampled working fluid. Embodiments are discussed with reference to an ICP-MS system. However, it should be understood that such embodiments may also be used with other types of spectrometric devices and are not limited only to an ICP-MS.

In some embodiments, the identified contaminant can then be traced back to a chamber component or material, based on at least the type of material identified. For example, contaminates including Fe, Ni, Cr can be traced back to SST materials. For example, Al, Y can be traced back to chamber components such as base materials or coating etc. Na, K, Ca from environment or handling. In such cases, a corrective action (e.g., such as part maintenance) can then be performed on the identified contamination source. In some embodiments, due to the in-situ nature of the analysis system, the identified contaminant can be used to effect a corrective action, as a process is occurring within a processing chamber or between processes.

Such a system and the embodiments discussed below provide advantages over conventional and known systems. Such a system and the embodiments discussed below provide additional functionalities that allow for in-situ contamination analysis, as a process is occurring within a processing chamber. Such a system enhances traceability and precision when identifying a contamination source. Such a system decreases and/or removes delays when performing contamination analysis. This may decrease a number of substrates that are processed by a chamber that is causing excess metal contamination, for example. Through such enhancements, such a system enhances uniformity in product yields, increasing final product quality, throughput times, and decreasing scrap and defective product cycles.

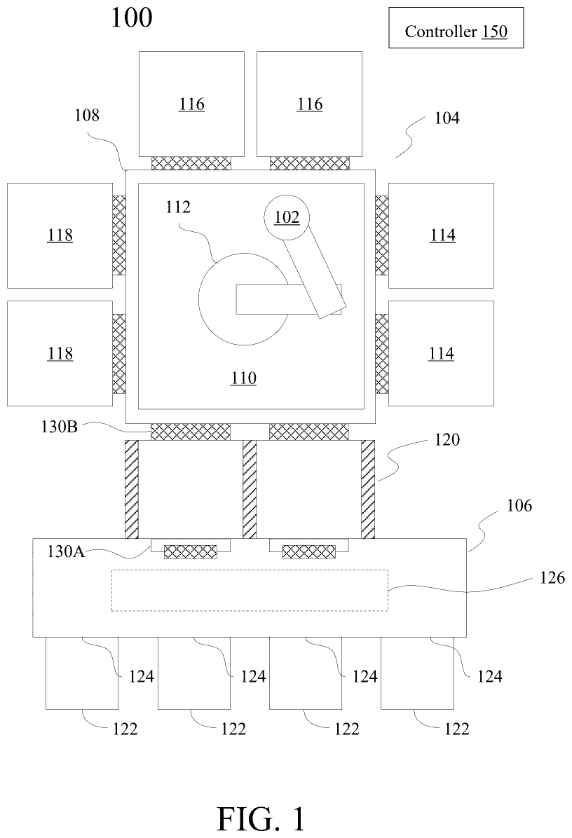

FIG. 1 illustrates a top-down view of an exemplary embodiment of a substrate manufacturing system, according to some embodiments of the present disclosure. It is noted that FIG. 1 is used for illustrative purposes, and that different component may be positioned in different location in relation to each view. In some embodiments, system 100 includes multiple processing chambers (e.g., chambers for substrate processing).

As seen, FIG. 1 illustrates a substrate manufacturing system 100 (also referred to as a “system,” “substrate processing system,” or “electronic device manufacturing system”) that is configured for substrate fabrication (e.g., for fabrication of semiconductor devices, displays, photovoltaic devices, etc.) in accordance with at least some embodiments of the disclosure. Substrates may be any suitably rigid, fixed-dimension, planar article, such as, e.g., a silicon-containing disc or wafer, a patterned wafer, a glass plate, or the like, suitable for fabricating electronic devices or circuit components thereon.

Electronic device manufacturing system 100 may include a process tool 104 (e.g., a mainframe) and a factory interface 106 (e.g., an EFEM) coupled to process tool 104. Process tool 104 may include a housing 108 having a transfer chamber 110 therein. Transfer chamber 110 includes one or more processing chambers (also referred to as process chambers) 114, 116, 118 disposed therearound and coupled thereto. Processing chambers 114, 116, 118 may be coupled to transfer chamber 110 through respective ports, such as slit valves or the like. In embodiments, a factory interface 106 may serve to transfer substrate to and from and substrate carriers 122 (at times referred to herein as “front opening unified pods (FOUPs)”).

Processing portion 104 may include a plurality of process chambers 114, 116, and 118, wherein specific and controlled substrate manufacturing processes occur. Transfer chamber 110 may house a transfer robot 112 including a substrate transfer mechanism such as an end effector 102 (“substrate transfer mechanism” and “end effector” will be used interchangeable moving forward in the disclosure) that may transport substrates. Transfer chamber 110 may be in transfer chamber housing 108. Load locks 120 may interface with both the processing portion 104 and the factory interface 106. Factory interface 106 may include a factory interface robot 126, for transferring substrates to and from the carriers 122 and the load locks 120. Factory interface may further include a plurality of load ports 124 for receiving carriers 122 carrying one or more substrates.

Transfer chamber 110 is generally maintained at vacuum pressure levels, while factory interface 106 is generally maintained at atmospheric pressure. In some embodiments, transfer chamber 110 and process chambers 114, 116, and 118, may be maintained at a vacuum level. Load locks 120 may alternate pressures between a vacuum level (e.g., when opened to transfer chamber 110) and atmospheric pressure (e.g., when opened to factory interface 106). The vacuum level for the transfer chamber 110 may range from about, e.g., 0.01 Torr (10 mTorr) to about 80 Torr. Other vacuum levels may be used.

Transfer chamber 110 may include a transfer chamber robot 112. In embodiments, the transfer chamber includes robot 112 may be configured to transfer the substrate from the load locks 120 to one or more of the plurality of processing chambers 114, 116, and 118 (also referred to as process chambers), or to one or more pass-through chambers (also referred to as vias), without vacuum break, i.e., while maintaining a vacuum pressure environment within the transfer chamber 110 and the plurality of processing chambers 114, 116, and 118.

Transfer chamber robot 112 may include one or multiple arms where each arm includes one or more end effectors at the end of each arm. The end effector may be configured to handle particular objects, such as wafers. Alternatively, or additionally, the end effector is configured to handle objects such as process kit rings. In some embodiments, transfer chamber robot 112 is a selective compliance assembly robot arm (SCARA) robot, such as a 2-link SCARA robot, a 3-link SCARA robot, a 4-link SCARA robot, and so on.

Processing chambers 114, 116, 118 may be adapted to carry out any number of processes on a substrates. A same or different substrate process may take place in each processing chamber 114, 116, 118. Examples of substrate processes include annealing, thermal processing, atomic layer deposition (ALD), chemical vapor deposition (CVD), etching, curing, pre-cleaning, metal or metal oxide removal, or the like. In one example, a CVD process is performed in one or both of process chambers 114, an etching process is performed in one or both of process chambers 116, and an annealing process is performed in one or both of process chambers 118. Other processes may be carried out on substrates therein. Processing chambers 114, 116, 118 may each include a substrate support assembly. The substrate support assembly may be configured to hold a substrate in place while a substrate process is performed.

A load lock 120 may also be coupled to housing 108 and transfer chamber 110. Load lock 120 may be configured to interface with, and be coupled to, transfer chamber 110 on one side and factory interface 106 on another side. Load lock 120 may have an environmentally controlled atmosphere that is changed from a vacuum environment (where substrates are transferred to and from transfer chamber 110) to an at or near atmospheric-pressure inert-gas environment (where substrates are transferred to and from factory interface 106) in some embodiments. In some embodiments, load lock 120 is a stacked load lock having a pair of upper interior chambers and a pair of lower interior chambers that are located at different vertical levels (e.g., one above another). In some embodiments, the pair of upper interior chambers are configured to receive processed substrates from transfer chamber 110 for removal from process tool 104, while the pair of lower interior chambers are configured to receive substrates from factory interface 106 for processing in process tool 104. In some embodiments, load lock 120 is configured to perform a substrate process (e.g., an etch or a pre-clean) on one or more substrates received therein.

In some embodiments, the load locks may be equipped with several sensors and/or integrated control systems for adding intelligent capabilities to the overall system. For example, such sensors may monitor various chamber parameters, such as temperature, pressure, cleanliness, etc., within the load lock chamber. Sensor data may then be relayed in real-time to a control system, which may responsively adjust conditions within the load lock to optimize substrate handling and processing. For instance, in some embodiments, a sensor of a load lock may detect a deviation in the vacuum level. In response, the control system might automatically activate pumps to restore pressure conditions. In a similar manner and example, temperature sensors may initiate cooling and/or heating elements to maintain the substrates at, or cause the substrates to reach, a predefined temperature range. In some embodiments, the number of load locks may be more or less than two but for illustration purposes only, two load locks 120 are shown with each load lock having a door (e.g., a slit valve) to connect it to the factory interface and a door to connect it to the transfer chamber 110. Load locks 120 may or may not be batch load locks. In embodiments, the metal contamination system (and/or an associated probe can also be used for monitoring load locks and other locations)

Factory interface 106 may be any suitable enclosure, such as, e.g., an Equipment Front End Module (EFEM). Factory interface 106 may be configured to receive substrates from substrate carriers 122 (e.g., Front Opening Unified Pods (FOUPs)) docked at various load ports 124 of factory interface 106.

In some embodiments, a factory interface robot 126 (shown dotted) may be configured to transfer substrates 102 between substrate carriers 122 (also referred to as containers) and load lock 120. The movement sequence may involve extracting a substrate from its FOUP 122 and securely placing it into one of the load locks through the corresponding load lock doors. In other and/or similar embodiments, factory interface 106 may be configured to receive replacement parts from replacement parts storage containers. In embodiments, factory interface robot 126 may be or include one or more robot arms and may be or include a SCARA robot. In some embodiments, factory interface robot 126 may have more links and/or more degrees of freedom than transfer chamber robot 112. Factory interface robot 126 may include an end effector on an end of each robot arm. The end effector may be configured to pick up and handle specific objects, such as wafers.

Alternatively, or additionally, the end effector may be configured to handle objects such as process kit rings. Any conventional robot type may be used for factory interface robot 126. Transfers may be carried out in any order or direction. In embodiments, factory interface 106 may be maintained in, e.g., a slightly positive-pressure non-reactive gas environment (using, e.g., nitrogen, other inert gasses, or air with controlled sub-component parameters as the non-reactive gas) in some embodiments.

Factory interface 106 may be configured with any number of load ports 124, which may be located at one or more sides of the factory interface 106 and at the same or different elevations.

Factory interface 106 may include one or more auxiliary components (not shown). The auxiliary components may include substrate storage containers, metrology equipment, servers, air conditioning units, etc. A substrate storage container may store substrates and/or substrate carriers (e.g., FOUPs), for example. Metrology equipment may be used to determine property data of the products that were produced by the electronic device manufacturing system 100. In some embodiments, factory interface 106 may include an upper compartment. The upper compartment may house electronic systems (e.g., servers, air conditioning units, etc.), utility cables, system controller 128, or other components. In some embodiments, the electronic systems, utility cables, etc. housed in the upper compartment include a processing chamber for microwave annealing for low thermal budget applications as described herein.

In some embodiments, transfer chamber 110, process chambers 114, 116, and 118, and/or load lock 120 may be maintained at a vacuum level. Electronics processing system 100 may include one or more vacuum ports that are coupled to one or more stations of electronic device manufacturing system 100. For example, first vacuum ports 130A may couple factory interface 106 to load locks 120. Second vacuum ports 130B may be coupled to load locks 120 and disposed between load locks 120 and transfer chamber 110.

In some embodiments, controller 150 (e.g., a tool and equipment controller, a tool cluster controller, etc.) may control various aspects of the cluster tool 100, e.g., gas pressure in the processing chambers, individual gas flows, spatial flow ratios, plasma power in various process chambers, temperature of various chamber components, radio frequency (RF) or electrical state of the processing chambers, and so on. The controller 150 may receive signals from and send commands to any of the components of the cluster tool 100, such as the robot 112 and 126, process chambers 114, 116, and 118, load locks 120, substrate support assemblies (e.g., including positional controls) of process chambers and/or load locks, cooling stations, slit valve doors, and/or one or more sensors (e.g., integrated in one or more substrate support assemblies of load locks and/or), and/or other processing components of the cluster tool 100.

The controller 150 may thus control the initiation and cessation of processing, may adjust a deposition rate and/or target layer thickness, may adjust process temperatures, may adjust a type or mix of deposition composition, may adjust an etch rate, may initiate processes on the load locks or processing chambers (e.g., such as cooling, heating, etc.), may determine when to remove substrates from cooling stations, may adjust coolant flow rate, may adjust coolant temperature, and the like. The controller 150 may further receive and process sensor measurement data (e.g., optical measurement data, vibration data, spectrographic data, particle detection data, temperature data, etc.) from various sensors (e.g., sensors integrated into substrate support assemblies of load locks and/or processing chambers) and make decisions based on such measurement data.

In various embodiments, the controller 150 may be and/or include a computing device such as a personal computer, a server computer, a programmable logic controller (PLC), a microcontroller, and so on. The controller 150 may include (or be) one or more processing devices, which may be general-purpose processing devices such as a microprocessor, central processing unit, or the like. More particularly, the processing device may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device may also be one or more special-purpose processing devices such as an application specific integrated circuit (ASIC), a field programmable gate array (FPGA), a digital signal processor (DSP), network processor, or the like. The controller 150 may include a data storage device (e.g., one or more disk drives and/or solid state drives), a main memory, a static memory, a network interface, and/or other components. The processing device of the controller 150 may execute instructions to perform any one or more of the methodologies and/or embodiments described herein. The instructions may be stored on a computer readable storage medium, which may include the main memory, static memory, secondary storage and/or processing device (during execution of the instructions). In some embodiments, controller 150 is a dedicated controller for the processing chambers.

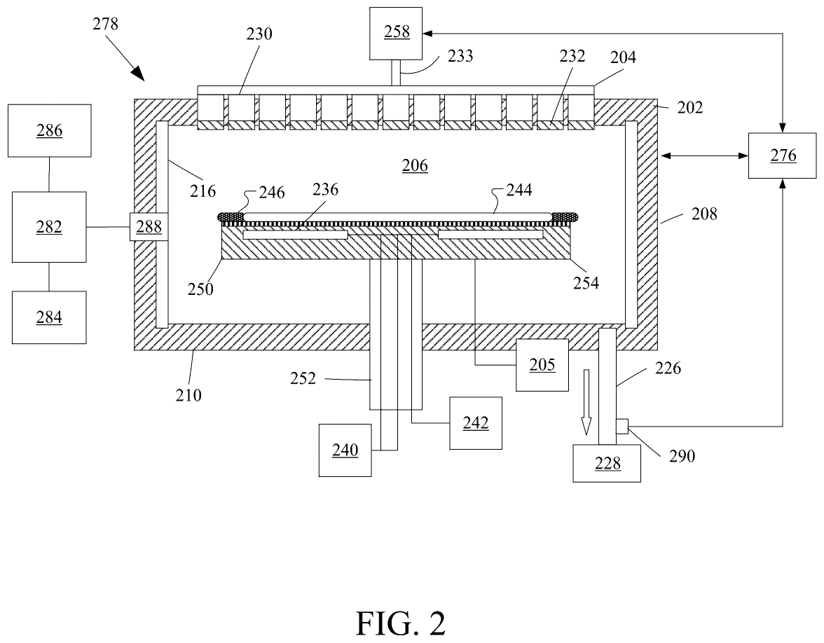

In one embodiment, a system controller 150 may be used to autonomously control and coordinate the proposed analysis subsystem (that may include a sampling chamber, a separate vacuum system, and ICP-MS system, as will be further described with respect to FIG. 2) that may be associated with a processing chamber (such as chambers 114, 116, 118). In embodiments, the controller 150 may be implemented in hardware, firmware, software, or a combination thereof. The system controller 150 may be configured to receive and process measurement data generated by one or more sensors (e.g., pressure sensors, ICP-MS system outputs, etc.) associated with both the analysis system, and manufacturing system at large. In embodiments, the controller may receive and process sensor measurement data from within processing chambers (e.g., of sensors associated with processing chamber 114, 116, 118). In embodiments, the sensor measurements may include temperature measurements, pressure measurements, particle measurements, spectrographic measurements, vibration measurements, accelerometer measurements, voltage measurements, current measurements, resistance measurements, time measurements, optical measurements (e.g., such as optical emission spectrometry measurements and/or reflectometry measurements), position measurements, humidity measurement, part health measurements, and/or other types of measurements. Some example measurements include a chamber pressure (e.g., which may be measured in mTorr), OES spectra measurements for one or more wavelengths or frequencies (e.g., for wavelengths of 3870 nm, 7035 nm, 775 nm, and so on), one or more substrate support/heater temperatures, one or more substrate temperatures, substrate presence, and so on. In some embodiments, some or all of these measurements may be combined to generate a feature vector that is input into a trained machine learning model of the system controller 150. In some embodiments, some or all of these measurements may be input into a rules-based engine that may determine one or more actions to perform based on the measurement(s) satisfying one or more criteria or rules.

Further embodiments of the system will now be provided. The controller 150 may include one or more rules-based engines and/or trained machine learning models for controlling and/or making decisions associated with the proposed analysis system. The one or more trained machine learning models may have been trained to receive sensor measurements from and/or associated with a processing chamber (and/or the ICP-MS) and to make a prediction, classification or determination about a contamination condition within a processing chamber. Each of the trained machine learning models may be associated with a different decision-making process for a processing chamber in embodiments. Alternatively, one or a few trained machine learning models may be associated with multiple decision-making processes for a load lock in embodiments.

In one embodiment, one or more of the trained machine learning models is a regression model trained using regression. Examples of regression models are regression models trained using linear regression or Gaussian regression. A regression model predicts a value of Y given known values of X variables. The regression model may be trained using regression analysis, which may include interpolation and/or extrapolation. In one embodiment, parameters of the regression model are estimated using least squares. Alternatively, Bayesian linear regression, percentage regression, leas absolute deviations, nonparametric regression, scenario optimization and/or distance metric learning may be performed to train the regression model.

In one embodiment, one or more of the trained machine learning models are decision trees, random forests, support vector machines, or other types of machine learning models.

In one embodiment, one or more of the trained machine learning models is an artificial neural network (also referred to simply as a neural network). The artificial neural network may be, for example, a convolutional neural network (CNN) or a deep neural network. In one embodiment, processing logic performs supervised machine learning to train the neural network.

Artificial neural networks generally include a feature representation component with a classifier or regression layers that map features to a target output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). The neural network may be a deep network with multiple hidden layers or a shallow network with zero or a few (e.g., 1-2) hidden layers. Deep learning is a class of machine learning algorithms that use a cascade of multiple layers of nonlinear processing units for feature extraction and transformation. Each successive layer uses the output from the previous layer as input. Neural networks may learn in a supervised (e.g., classification) and/or unsupervised (e.g., pattern analysis) manner. Some neural networks (e.g., such as deep neural networks) include a hierarchy of layers, where the different layers learn different levels of representations that correspond to different levels of abstraction. In deep learning, each level learns to transform its input data into a slightly more abstract and composite representation.

Some trained machine learning models of an autonomous substrate support engine 152 use all sensor measurements generated by a processing chamber and/or associated devices. Some trained machine learning models of controller 150 may use a subset of generated sensor measurements. For example, some trained machine learning models may operate on data from an ICP-MS, where the ICP-MS may generate such data based on sampling of gases from a process chamber in-situ during execution of a process in the process chamber.

Controller 150 may be operatively connected to a server (not shown). The server may be or include a computing device that operates as a factory floor server that interfaces with some or all tools in a fabrication facility. The server may perform training to generate the trained machine learning models, and may send the trained machine learning models to controller 150. Alternatively, the machine learning models may be trained on controller 150.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

FIG. 2 illustrates a schematic view of an exemplary contaminant analysis system and exemplary substrate processing chamber of FIG. 1, according to some embodiments of the present disclosure.

Referring to FIG. 2, the vacuum sampling system may be attached to a processing chamber 278 (which may correspond, or be similar, to processing chambers 114, 116, 118, as discussed with respect to FIG. 1), which may perform plasma-based processes (e.g., such as etching, cleaning, deposition, etc.), in embodiments. Alternatively, processing chamber may perform non-plasma processes e.g., such as annealing, polishing, etc.

In cases where processing chamber 278 performs plasma processes, the processing chamber 278 may be coupled to a plasma source 258 via one or more gas delivery lines 233. The processing chamber 278 may be, for example, a plasma etch reactor, a deposition chamber, an ashing chamber, etc. The processing chamber may be suitable for an etching operation, a deposition operation, a chamber cleaning operation, a plasma treatment operation, or any other type of operation typical of a semiconductor manufacturing facility. In an embodiment, one or more substrates (e.g., wafers) 244 may be provided within the processing chamber 278. In an embodiment, the processing chamber 278 may be maintained at a pressure suitable for the target operation. In a particular embodiment, the pressure may be between approximately 1 Torr and approximately 200 Torr.

The processing chamber 278 and/or plasma source 258 may be connected to a controller 276, which may control processing of the plasma source 258 and/or processing chamber 278 (e.g., by controlling set points, loading recipes, and so on). One or more flow sensors and/or pressure sensors may be connected to the gas delivery line(s) to detect gas flow characteristics in some embodiments.

In embodiments, the plasma source 258 is a remote plasma source (RPS) that generates plasma at a remote location and delivers the externally generated plasma to the processing chamber. Alternatively, the processing chamber may include an integrated plasma source (not shown) that can generate plasma within the processing chamber.

Processing chamber 278 includes a substrate support assembly 250, according to some embodiments. Substrate support assembly 250 includes a puck 254 (e.g., may include an electrostatic chuck (ESC)). The puck 254 may perform chucking operations, e.g., vacuum chucking, electrostatic chucking, etc. Substrate support assembly 250 may further include base plate, cooling plate and/or insulator plate (not shown).

In embodiments, processing chamber 278 includes chamber body 202 and lid 204 that encloses an interior volume 206. Chamber body 202 may be fabricated from aluminum, stainless steel, or any other suitable material. Chamber body 202 generally includes sidewalls 208 and a bottom 210. An outer liner 216 may be disposed adjacent to sidewalls 208, e.g., to protect chamber body 202. Outer liner 216 may be fabricated and/or coated with a plasma or halogen-containing gas resistant material. Outer liner 216 may be fabricated from or coated with aluminum oxide. Outer liner 216 may be fabricated from or coated with yttria, yttrium alloy, oxides thereof, etc.

An exhaust port may be defined in chamber body 202, and may couple interior volume 206 to an exhaust line 226 that in turn couples to a pump system 228. Pump system 228 may include one or more pumps, valves, lines, manifolds, tanks, etc., utilized to evacuate and regulate the pressure of interior volume 206. In embodiments, a pressure sensor and/or flow sensor 290 is disposed in or on the exhaust line. The pressure sensor and/or flow sensor 290 may be used to measure a pressure and/or a gas flow rate at or near pump system 228. Additionally, or alternatively, one or more other pressure sensors and/or flow sensors may be disposed at other locations at the exhaust line 226, in the processing chamber 278 and/or at gas delivery line(s) 233.

Lid 204 may be supported on sidewall 208 of chamber body 202. Lid 204 may be openable, allowing access to interior volume 206. Lid 204 may provide a seal for processing chamber 278 when closed. Plasma source 258 may be coupled to processing chamber 278 to provide process, cleaning, backing, flushing, etc., gases and/or plasmas to interior volume 206 through gas distribution assembly 230. Gas distribution assembly 230 may be integrated with lid 204 in embodiments.

Examples of processing gases that may be used in processing chamber 278 include halogen-containing gases, such as C2F6, SF6, SiCl4, HBr, NF3, CF4, CHF3, CH2F3, Cl2 and SiF4. Other reactive gases may include O2 or N2O. Non-reactive gases may be used for flushing or as carrier gases, such as N2, He, Ar, etc. (Metal precursors, such as aluminum, hafnium, cobalt, titanium, tantalum, tungsten, and zirconium, and their residue may also be used). Gas distribution assembly 230 (e.g., showerhead) may include multiple inlets 232 on the downstream surface of gas distribution assembly 230. Inlets 232 may direct gas flow to the surface of substrate 244. In some embodiments, gas distribution assembly may include a nozzle (not pictured) extended through a hold in lid 204. A seal may be made between the nozzle and lid 204. Gas distribution assembly 230 may be fabricated and/or coated by a ceramic material, such as silicon carbide, yttrium oxide, etc., to provide resistance to processing conditions of processing chamber 278.

Substrate support assembly 250 is disposed in interior volume 206 of processing chamber 278 below gas distribution assembly 230. Substrate support assembly 250 may hold a substrate 244 during processing. An inner liner (not shown) may be coated on the periphery of substrate support assembly. The inner liner may share features (e.g., materials of manufacture, function, etc.) with outer liner 216.

Substrate support assembly 250 may include supporting pedestal 252, insulator plate, base plate, cooling plate, and puck 254. Puck 254 may include electrodes 236 for providing one or more functions. Electrodes 236 may include chucking electrodes (e.g., for securing substrate 244 to an upper surface of puck 254), heating electrodes, RF electrodes for plasma control, etc.

Protective ring 246 may be disposed over a portion of puck 254 at an outer perimeter of puck 254. Puck 254 may be coated with a protective layer (not shown).

Puck 254 may further include multiple gas passages such as grooves, mesas, and other features that may be formed in an upper surface of puck 254. Gas passages may be fluidly coupled to a gas source 205. Gas from gas source 205 may be utilized as a heat transfer or backside gas, may be utilized for control of one or more lift pins of puck 254, etc. Multiple gas sources may be utilized (not shown). Gas passages may provide a gas flow path for a backside gas such as He via holes drilled in puck 254. Backside gas may be provided at a controlled pressure into gas passages to enhance heat transfer between puck 254 and substrate 244.

Puck 254 may include one or more clamping electrodes. The clamping electrodes may be controlled by chucking power source 240. Clamping electrodes may further couple to one or more RF power sources through a matching circuit for maintaining a plasma formed from process and/or other gases within processing chamber 278. The RF power sources may be capable of producing an RF signal having a frequency from about 50 kilohertz (kHz) to about 3 gigahertz (GHz) and a power of up to about 10,000 Watts. Heating electrodes of puck 254 may be coupled to heater power source 242.

Controller 276 may control one or more parameters and/or set points of the plasma source 258 and/or processing chamber 278. (Controller 276 may correspond, or be similar, to controller 150 as discussed with respect to FIG. 1). System controller 276 can be and/or include a computing device such as a personal computer, a server computer, a programmable logic controller (PLC), a microcontroller, and so on. System controller 276 can include one or more processing devices, which can be general-purpose processing devices such as a microprocessor, central processing unit, or the like. More particularly, the processing device can be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device can also be one or more special-purpose processing devices such as an application specific integrated circuit (ASIC), a field programmable gate array (FPGA), a digital signal processor (DSP), network processor, or the like. System controller 276 can include a data storage device (e.g., one or more disk drives and/or solid-state drives), a main memory, a static memory, a network interface, and/or other components.

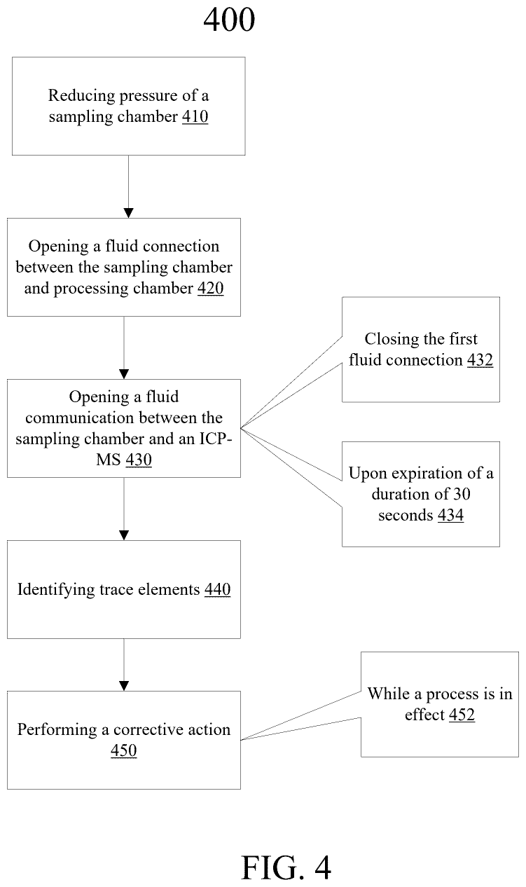

System controller 276 can execute instructions to perform any one or more of the methodologies and/or embodiments described herein. The instructions can be stored on a computer readable storage medium, which can include the main memory, static memory, secondary storage and/or processing device (during execution of the instructions). In embodiments, execution of the instructions by system controller 276 causes system controller to perform the methods of FIG. 4.

In some embodiments, substrate 244 may be made out of one or more materials including Silicon, Germanium, Gallium Arsenide (GaAs), silicon dioxide (SiO₂ or silica), Indium Phosphide (InP), Silicon Germanium (SiGe), Silicon Carbide (SiC), Gallium Nitride (GaN), glass, or any one or more materials commonly used within electronic device manufacturing systems. In some embodiments, substrate 244 may be formed from one material. In other embodiments, substrate 244 may be formed from a homogenous mixture of materials. In other embodiments, substrate 244 may be made out of one or more stacked layers of one or more differing materials.

Any of the components of the processing chamber may degrade over time, which may cause the components to shed metal particles and/or cause metal contamination of processed substrates.

In some embodiments, the chamber may be connected to a sampling chamber 284, which may or may not be operatively connected to an inductively coupled plasma mass spectrometer (ICP-MS) 286 and/or other type of metrology tool, (e.g., another type of spectrometric device such as a TOF-MS tool, or a GC-MS tool). In embodiments, the sampling chamber 284 and/or ICP-MS 286 may be fluidly connected to an interior of the processing chamber via a port 288 and valve 282. In embodiments, such a fluid connection or communication may allow gasses from within the chamber, including gaseous trace elements or compounds, to be delivered to both the sampling chamber and ICP-MS.

The sampling chamber 284 may be similar in size to, or of smaller size than, the processing chamber. In embodiments, the sampling chamber may be of any volumetric shape e.g., cylindrical, cubic, spherical, etc., so as to load and contain a gaseous sample from the processing chamber.

In embodiments, the sampling chamber may include an inner volume of anywhere from 2 to 10 liters (e.g., 6 liters).

In embodiments, the sampling chamber may include a heating element and/or cooling element (e.g., internally or externally) such as a heat lamp, convective heat source, etc. In embodiments, such an element may be used to modulate, maintain, or control the temperature of a gas or substance within the chamber.

In some embodiments, the sampling chamber 284 is portable. In some embodiments, the sampling chamber 284 is removable and/or swappable. For example, the sampling chamber 284 may be used to collect a sample of air-borne particles from a process chamber 278, and transported to a lab for further analysis. For example, metal species and/or nanoparticles may be measured using a metrology tool that performs ICP-MS and/or sp-ICP-MS that is not connected to the process chamber and/or associated equipment being tested. Similarly, a metrology tool that performs time-of-flight mass spectrometry (TOF-MS) may be attached to the sampling chamber 284 and used to measure organic and/or inorganic species. Additionally, a metrology tool that performs gas chromatography-mass spectrometry (GC-MS) may be attached to the sampling chamber 284 for performing of further analysis. Accordingly, after a sample of airborne particles is collected from an interior volume 206 of process chamber 278 in the sampling chamber 284, the sampling chamber 284 may then be disconnected from the equipment from which the sample was collected and then connected to one or more types of sensors or metrology tools for off-line measurements. In embodiments, a gas such as N2 and/or Ar may be used for force captured particles (e.g., a sample) out of the sampling chamber and into a metrology tool such as an ICP-MS tool, a TOF-MS tool, or a GC-MS tool. This increases a flexibility of the vacuum sampling system in embodiments.

In some embodiments, multiple sampling chambers (e.g., sample tanks) can be used to take samples of process chamber 278, one at a time. A first sampling chamber may be attached to the valve 282 and may be used to collect a sample. The first sampling chamber may be disconnected, and a second sampling chamber may be attached to the valve 282 and may be used to collect another sample. The different sampling chambers may then be connected to different metrology tools to perform different tests on the samples.

In some embodiments, sampling chamber 284 is a stainless steel tank. The sampling chamber may have a volume of, for example, 6 liters, 4 liters, 8 liters, 10 liters, and so on. The sampling chamber may have a vacuum pressure of 1 Torr to about E-5 Torr (e.g., 0.02 Torr) in embodiments while it contains a sample. In embodiments, the sampling chamber may maintain a target pressure for days after being removed from the equipment under test.

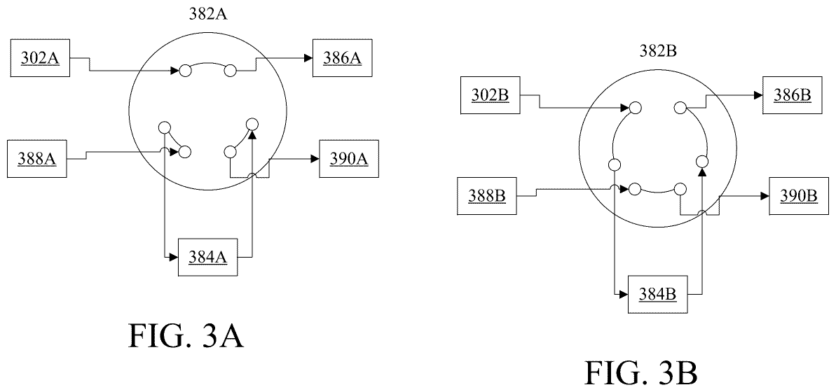

In embodiments, the valve 282 may be designed and/or attached to the port, ICP-MS, and sampling chamber 284 in such a way as to effect an analysis process (which will be further seen and described with respect to FIGS. 3A-B).

In embodiments, during a phase of the analysis process, the valve may be switched into a position to lower or reduce the pressure within the sampling chamber 284. In some embodiments, the pressure within the sampling chamber may be lowered to 0.02 Torr or 0.015 Torr. In embodiments of such a phase, the valve may be switched into a position to open a first fluid connection between the sampling chamber 284 and a vacuum source (not shown in FIG. 2). In embodiments of such a phase, the sampling chamber may be isolated from the main processing chamber. In alternate embodiments, the pressure may be reduced while the sampling chamber is in fluid communication with the processing chamber.

In embodiments, during a subsequent phase of the analysis process (or in combination with the above-described phase), the sampling chamber may be loaded, or saturated, with working gas from the processing chamber. In embodiments of such a phase, the valve 282 may be switched into a position to ingress fluid to the sampling chamber directly from an interior volume of the processing chamber. In embodiments of such a phase, fluid may ingress into the sampling chamber as a process is occurring within the processing chamber. In embodiment of such a phase, the sampling chamber and processing chamber may be fluidly connected, and isolated from all other components.

In embodiments, the pressure level within the processing chamber during such a phase may be between 2 and 10 Torr.

In embodiments, such a phase may last for a duration of anywhere between 1 second to 30 seconds. In embodiments, such a phase may last for a duration of up to 5 minutes.

In embodiments, during a subsequent phase of the analysis process, the collected, or loaded gas from the processing chamber may then be transferred to the ICP-MS for analysis. In embodiments of such a phase, the valve 282 may be switched into a position to transfer fluid from the sampling chamber to the ICP-MS. In embodiments of such a phase, the valve may be switched to fluidly connect the sampling chamber to a gas source, that may provide a carrier gas to facilitate transfer for the sampling chamber contents (e.g., collected fluid) to be transferred to the ICP-MS.

In some embodiments, once the carrier gas delivers the contents of the sampling chamber to the ICP-MS, the ICP-MS may then be used for detecting, identifying, and quantifying trace elements, including metallic nanoparticles or particles or precursors within a gas or carrier gas. In some embodiments, the sampled or collected gas may include particles of any metal or metallic oxides, such as aluminum, boron, calcium, chromium, cobalt, copper, iron, lithium, magnesium, nickel, potassium, sodium, tin, tantalum, titanium, tungsten, zinc, zirconium, sulfur, barium, beryllium, bismuth, cadmium, gallium, germanium, hafnium, lead, manganese, molybdenum, antimony, strontium, yttrium, etc., or aluminum oxide, yttrium oxide, silicon oxide, copper oxide, or iron oxide, etc.

In embodiments, the ICP-MS system may operate by ionizing the delivered gas sample with a plasma torch. In embodiments, the gas sample may first nebulized, converting it into an aerosol. The aerosol may then be transported into the plasma torch (e.g., an argon plasma torch) of the ICP-MS. Upon entering the plasma, the constituent elements of the aerosol may ionize. The thermal energy provided by the torch may be sufficient to ionize any element present in the sample.

After ionization, the mass spectrometer may identify and quantify the ions via a series of quadrupoles. Through such a mechanism, and for each targeted element or isotope, the ICP-MS system can quantify a number of ions, which may directly correlate to the concentration of that element within the sample. Such a mechanism may allow for the detection of trace amounts of metal particles in the gas, even at very low concentrations.

To identify particles of smaller size (e.g., nanoparticles), in embodiments, the ICP-MS may include a component for advanced detection, or single particle identification and detection (i.e., a single particle ICP-MS, or “sp-ICP-MS”). In embodiments, the sp-ICP-MS component, may be integrated into the ICP-MS and leverage the components (e.g., plasma torch, nebulizer, quadrupoles, etc.) of the ICP-MS. In alternate embodiments, the sp-ICP-MS may be separate from the ICP-MS, and include separate components. In some embodiments, and analysis may be performed sequentially with respect to the ICP-MS system (or in either order).

Thus, in embodiments, an integrated sp-ICP-MS may extend the capabilities of the standard ICP-MS by leveraging the existing functions and components of the standard ICP-MS. The combined ICP-MS system (including components for single particle identification) may perform bulk elemental analysis and single-particle analysis. For instance, the integrated system may quantify total metal concentrations in the gaseous sample (using the ICP-MS portion) and then quantify metallic nanoparticles in the same sample (using sp-ICP-MS portion).

Once a level of contamination (e.g., a level of metallic particles and/or nanoparticles) has been identified, the identified contaminant can then be traced back to a chamber component or material, based on at least the type of material identified. In such cases, a corrective action (e.g., such as part maintenance) may then be performed on the identified contamination source.

In some embodiments, the corrective action may be effected after the process has terminated. In some embodiments, due to the in-situ nature of the analysis system, the identified contaminant and associated data can be used to effect a corrective action, as a process is occurring within a processing chamber. For instance, should a particularly high-level of contaminant or contamination be detected, the process may be prematurely terminated. In some cases, processing parameters or chamber conditions (e.g., temperatures, purging time, etc.) may be adjusted.

In some embodiments, the analysis system and valve may be designed or implemented in such a way as to include more or less phases than those described above. In some embodiments, the valve may be any type of valve that may be used to switch, isolate, or open, fluid connections between separate lines, components, and/or chambers. In embodiments, the valve 282 may be controlled by the system controller (e.g., controller 276, or 150, as was described with respect to FIG. 1).

In some embodiments, the analysis process may be run more than once during a processing chamber process. For instance, during a processing chamber process with a duration of 10 minutes, the analysis process may be run for one minute, at every minute, for a total of 10 cycles of the analysis process. In some embodiments, any number of cycles, or runs, or the analysis process may be performed during a single operation.

In some embodiments, the data gathered from the analysis process may be further processed, or categorized, and used as input for a software or computer model. In some instances, contamination data gathered from multiple analysis processes may be gathered and processed, and be used to train a machine learning, artificial intelligence, or similar model.

FIG. 3A illustrates a first exemplary configuration of the contaminant analysis system of FIG. 2, according to some embodiments of the present disclosure.

In some embodiments, elements within FIG. 3A may correspond, or be similar, to elements as discussed with respect to FIG. 2. For instance, port 388A, valve 382A, sampling chamber 384A, and ICP-MS 386A may correspond, or be similar to port 288, valve 282, sampling chamber 284, and ICP-MS 286 as seen and described with respect to FIG. 2. Accordingly, such components may incorporate and augment at least the embodiments discussed with respect to FIG. 2.

FIG. 3A may represent a first configuration of the valve 382A. In embodiments, in the first configuration, a first fluid connection and a second fluid connection may be formed. In embodiments, a fluid connection may be formed from the port 388A and a respective processing chamber, to a sampling chamber 384A, and to a vacuum source 390A.

In some embodiments of the configuration seen in FIG. 3A, the vacuum source 390A may decrease the pressure within the sampling chamber. In some embodiments, the decreased pressure may draw fluid from the processing chamber, or fluid may egress from the processing chamber, through port 388A, to sampling chamber 384A.

In some embodiments, the fluid ingressed into the sampling chamber may contain trace elements of metal contamination. In embodiments, the fluid may be a gas that contains nanoparticles or metallic particles originating from a component of the processing chamber.

In some embodiments of the configuration seen in FIG. 3A, a fluid connection may be formed from a gas source 302A and ICP-MS 386A. In some embodiments the gas source 302A may include a valve that may be closed in such a configuration. The ICP-MS may be in an off, or a non-functioning state.

As discussed above, in some embodiments the ICP-MS is not connected to the sampling chamber during sampling. In such embodiments, after collecting a sample the sampling chamber may be removed from a system under test and may be connected to a metrology tool such as an ICP-MS tool for analysis of the collected sample. In some embodiments, the ICP-MS tool or another metrology tool is connected to the sampling chamber. However, a technician may remove the sampling chamber after it has been filled with a sample. The sampling chamber may then be connected to a metrology device for analysis of the sample in the sampling chamber. To enable the sampling chamber to be removable, in some embodiments a detachable connection is provided between port 388A and valve 382A, between an input of sampling chamber 384A and valve 382A, between an output of sampling chamber 384A and valve 382A, and/or between ICP-MS 386A (or other metrology tool) and valve 382A.

As seen with respect to FIG. 3A, in some embodiments, the valve, and analysis system at large may be configured to isolate the sampling chamber from the ICP-MS. In some embodiments, fluid may ingress fluid into the sampling chamber, in anticipation of a second phase and configuration. In embodiments, the second phase and configuration may identify and measure trace elements (e.g., metallic particles) within the fluid.

FIG. 3B illustrates a second exemplary configuration of the contaminant analysis system of FIG. 2, according to some embodiments of the present disclosure.

In some embodiments, elements within FIG. 3B may correspond, or be similar, to elements as discussed with respect to FIGS. 2-3A. For instance, gas source 302B, port 388B, valve 382B, sampling chamber 384B, vacuum source 390B, and ICP-MS 386B may correspond, or be similar to gas source 302A, port 288 and 388A, valve 282 and 382A, sampling chamber 284 and 384A, vacuum source 390A, and ICP-MS 386A as seen and described with respect to FIGS. 2-3A. Accordingly, such components may incorporate and augment at least the embodiments discussed with respect to FIGS. 2-3A.

FIG. 3B may represent a second, or secondary, configuration of the valve 382B. In embodiments, in the second configuration, a first fluid connection and a second fluid connection may be formed. In embodiments, a fluid connection may be formed from the gas source 302B, to sampling chamber 348B, to ICP-MS 386B. In embodiments, a fluid connection may be formed from the port 388B to the vacuum source 390B. In embodiments, either fluid connection may be considered the first or second. In embodiments, the two fluid connections may be isolated, or separate.

In some embodiments the gas source 302A may include a valve that may be closed or opened in such a configuration. In some embodiments of the configuration seen in FIG. 3B, the gas source 302B may provide a carrier gas (e.g., argon gas), to flush or deliver the components and fluid within sampling chamber 384B to the ICP-MS 386. Upon delivery to the ICP-MS, analysis may be done on the delivered fluid, to identify trace elements (e.g., metal, or metallic particles) within the fluid.

As seen with respect to FIG. 3B, in some embodiments, the valve, and analysis system at large may be configured to isolate the gas source, the sampling chamber and the ICP-MS.

FIG. 4 is a flow diagram of an example method of performing in-situ analysis of metal species within a semiconductor processing chamber, in accordance with one embodiment of the present disclosure.

Method 400 may be performed by processing logic that can include hardware (circuitry, dedicated logic, etc.), software (e.g., instructions run on a processing device), or a combination thereof. In one implementation, some, or all of the operations of method 400 can be performed by one or more components of system 100 of FIG. 1.

At block 410, processing logic can reduce the pressure of a sampling chamber. In some embodiments, processing logic can reduce a pressure within a sampling chamber;

At block 420, processing logic can open a fluid connection between the sampling chamber and processing chamber. In some embodiments, processing logic can open, while a process is in effect within a processing chamber connected to the sampling chamber, a first fluid connection from the sampling chamber to the processing.

At block 430, processing logic can open a fluid communication between the sampling chamber and an ICP-MS. In some embodiments, processing logic can open a second fluid connection from the sampling chamber to an inductively coupled plasma mass spectrometer (ICP-MS) such that the fluid egresses from the sampling chamber into the ICP-MS.

At block 432, processing logic can close the first fluid connection.

At block 434, processing logic can open the second fluid connection and close the first fluid connection upon expiration of 30 seconds. In some embodiments, processing logic can open the second fluid connection and close the first fluid connection upon expiration of 30 seconds. In some embodiments, processing logic can transition a valve configured to transition from the first setting to the second setting upon expiration of a duration of 30 seconds.

At block 440, processing logic can identify trace elements. In some embodiments, processing logic can identify, via the ICP-MS, a presence of trace elements within the fluid.

At block 450, processing logic can perform a corrective action. In some embodiments, processing logic can, responsive to the identified presence of trace elements, perform a corrective action associated with the processing chamber.

At block 452, processing logic can perform a corrective action while a process is in effect. In some embodiments, processing logic can perform the corrective action while the process is in effect. In some embodiments the corrective action can include one of terminating the process or adjusting a parameter of the process.

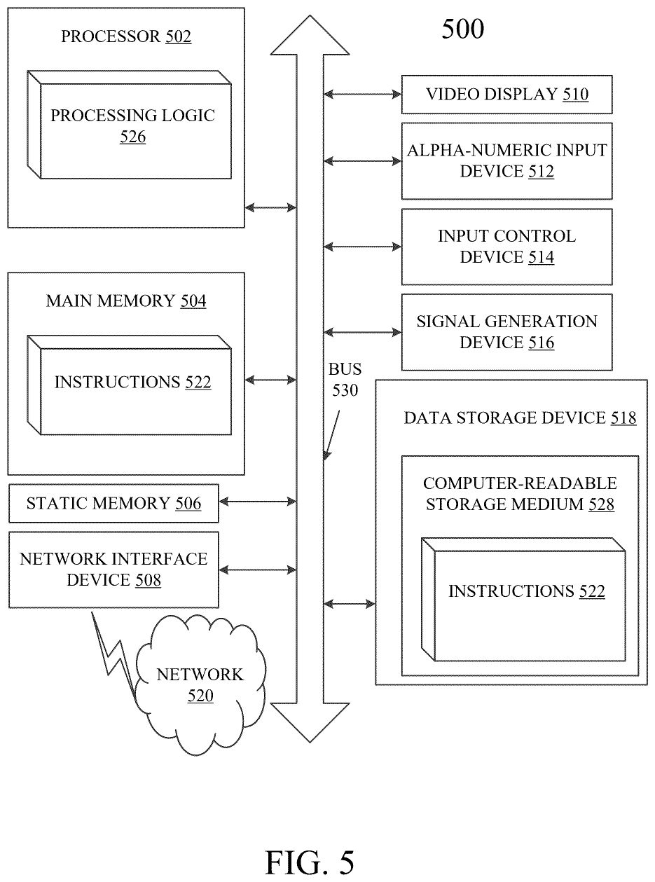

FIG. 5 illustrates an embodiment of a diagrammatic representation of a computing device associated with a substrate manufacturing system, in according to some embodiments of the present disclosure.

In one implementation, FIG. 5 illustrates a processing device 500 that may be a part of any computing device associated with any of the above-described figures, or any combination thereof. Example processing device 500 may be connected to other processing devices in a LAN, an intranet, an extranet, and/or the Internet. The processing device 500 may be a personal computer (PC), a set-top box (STB), a server, a network router, switch or bridge, or any device capable of executing a set of instructions (sequential or otherwise) that specify actions to be taken by that device. Further, while a single example processing device is illustrated, the term “processing device” shall also be taken to include any collection of processing devices (e.g., computers) that individually or jointly execute a set (or multiple sets) of instructions to perform any one or more of the methods discussed herein.

Example processing device 500 may include a processor 502 (e.g., a CPU), a main memory 504 (e.g., read-only memory (ROM), flash memory, dynamic random access memory (DRAM) such as synchronous DRAM (SDRAM), etc.), a static memory 506 (e.g., flash memory, static random access memory (SRAM), etc.), and a secondary memory (e.g., a data storage device 518), which may communicate with each other via a bus 530.

Processor 502 represents one or more general-purpose processing devices such as a microprocessor, central processing unit, or the like. More particularly, processor 502 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 502 may also be one or more special-purpose processing devices such as an application specific integrated circuit (ASIC), a field programmable gate array (FPGA), a digital signal processor (DSP), network processor, or the like. In accordance with one or more aspects of the present disclosure, processor 502 may be configured to execute instructions (e.g. instructions 522 may include a computing subsystem as seen at least in within the controller of FIG. 1). In embodiments, instructions 522 include instructions for a performing the method of FIG. 4.

Example processing device 500 may further comprise a network interface device 508, which may be communicatively coupled to a network 520. Example processing device 500 may further comprise a video display 510 (e.g., a liquid crystal display (LCD), a touch screen, or a cathode ray tube (CRT)), an alphanumeric input device 512 (e.g., a keyboard), an input control device 514 (e.g., a cursor control device, a touch-screen control device, a mouse), and a signal generation device 516 (e.g., an acoustic speaker).

Data storage device 518 may include a computer-readable storage medium (or, more specifically, a non-transitory computer-readable storage medium) 528 on which is stored one or more sets of executable instructions 522. In accordance with one or more aspects of the present disclosure, executable instructions 522 may comprise executable instructions.

Executable instructions 522 may also reside, completely or at least partially, within main memory 504 and/or within processor 502 during execution thereof by example processing device 500, main memory 504 and processor 502 also constituting computer-readable storage media. Executable instructions 522 may further be transmitted or received over a network via network interface device 508.

While the computer-readable storage medium 528 is shown in FIG. 5 as a single medium, the term “computer-readable storage medium” should be taken to include a single medium or multiple media (e.g., a centralized or distributed database, and/or associated caches and servers) that store the one or more sets of operating instructions. The term “computer-readable storage medium” shall also be taken to include any medium that is capable of storing or encoding a set of instructions for execution by the machine that cause the machine to perform any one or more of the methods described herein. The term “computer-readable storage medium” shall accordingly be taken to include, but not be limited to, solid-state memories, and optical and magnetic media.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily rely on the particular order shown, or sequential order, to achieve desirable results. In some cases, multitasking and parallel processing may be advantageous.

What is claimed is:

1. A method comprising:
reducing a pressure within a sampling chamber;
opening, while a process is in effect within a processing chamber connected to the sampling chamber, a first fluid connection from the sampling chamber to the processing chamber such that a fluid ingresses from the processing chamber into the sampling chamber;
closing the first fluid connection;
opening a second fluid connection from the sampling chamber to a spectrometric device such that the fluid egresses from the sampling chamber into the spectrometric device;
identifying, via the spectrometric device, a presence of trace elements within the fluid; and
responsive to the identified presence of trace elements, performing a corrective action associated with the processing chamber.

2. The method of claim 1, wherein the pressure within the sampling chamber is reduced to a value of 0.02 Torr.

3. The method of claim 1, wherein a pressure within the processing chamber is within 1-10 Torr.

4. The method of claim 1, wherein the fluid comprises a gas comprising the trace elements.

5. The method of claim 1, wherein the trace elements identified via the spectrometric device comprise at least one of aluminum, boron, calcium, chromium, cobalt, copper, iron, lithium, magnesium, nickel, potassium, sodium, tin, titanium, tungsten, zinc, zirconium, sulfur, barium, beryllium, bismuth, cadmium, gallium, germanium, hafnium, lead, manganese, molybdenum, antimony, strontium, or yttrium.

6. The method of claim 1, wherein the trace elements identified via the spectrometric device comprise nanoparticles of at least one of aluminum oxide, yttrium oxide, silicon oxide, copper oxide, or iron oxide.

7. The method of claim 1, wherein the corrective action is performed while the process is in effect and comprises one of terminating the process or adjusting a parameter of the process.

8. A system comprising:
a processing chamber;
a spectrometric device to identify a presence of trace elements within a fluid from the processing chamber; and
a valve configured to connect the processing chamber to a sampling chamber in a first valve setting to cause the fluid to ingress from the processing chamber into the sampling chamber and to connect the sampling chamber to the spectrometric device in a second valve setting to cause the fluid to egress from the sampling chamber into the spectrometric device.

9. The system of claim 8, wherein the valve is configured to transition from the first setting to the second setting upon expiration of a duration of 30 seconds.

10. The system of claim 8, wherein the valve is configured to selectively connect the sampling chamber and either the processing chamber or the spectrometric device, and wherein the spectrometric device is an inductively coupled plasma mass spectrometer.

11. The system of claim 8, wherein in the first valve setting the sampling chamber is connected to a vacuum system configured to reduce a pressure within the sampling chamber to a level lower than a pressure within the processing chamber such that fluid ingresses into the sampling chamber.

12. The system of claim 8, wherein in the second valve setting the sampling chamber is connected to a gas source which provides a flow of gas to cause fluid within the sampling chamber to ingress into the spectrometric device.

13. The system of claim 8, wherein the fluid comprises a gas comprising trace elements.

14. The system of claim 13, wherein the trace elements identified via the spectrometric device comprise at least one of aluminum, boron, calcium, chromium, cobalt, copper, iron, lithium, magnesium, nickel, potassium, sodium, tin, titanium, tungsten, zinc, zirconium, sulfur, barium, beryllium, bismuth, cadmium, gallium, germanium, hafnium, lead, manganese, molybdenum, antimony, strontium, or yttrium.

15. The system of claim 13, wherein the trace elements identified via the spectrometric device comprise nanoparticles of at least one of aluminum oxide, yttrium oxide, silicon oxide, copper oxide, or iron oxide.

16. The system of claim 8, wherein a corrective action is performed while a process is in effect within the processing chamber and comprises one of terminating the process or adjusting a parameter of the process.

17. The system of claim 8, wherein the sampling chamber is removable from the system.

18. A non-transitory computer readable storage medium comprising instructions that, when executed by a processing device, causes the processing device to perform operations comprising:
reducing a pressure within a sampling chamber;
opening, while a process is in effect within a processing chamber connected to the sampling chamber, a first fluid connection from the sampling chamber to the processing chamber such that a fluid ingresses from the processing chamber into the sampling chamber;
closing the first fluid connection;
opening a second fluid connection from the sampling chamber to an inductively coupled plasma mass spectrometer (ICP-MS) such that the fluid egresses from the sampling chamber into the ICP-MS;
identifying, via the ICP-MS, a presence of trace elements within the fluid; and
responsive to the identified presence of trace elements, performing a corrective action associated with the processing chamber.

19. The non-transitory computer readable storage medium of claim 18, wherein a valve is configured to close the first fluid connection and open the second fluid connection upon expiration of a duration of 30 seconds.

20. The non-transitory computer readable storage medium of claim 18, wherein a valve is configured to selectively connect the sampling chamber and either the processing chamber or the ICP-MS.
